(19) United States
(12) Patent Application Publication
(10) Pub. No.: US 2024/0280556 A1
(43) Pub. Date: Aug. 22, 2024

(54) ELECTROCONDUCTIVITY CAPACITIVE SENSOR FOR IN SITU SOIL ANALYSIS

(71) Applicant: CHRYSALABS INC., Montreal (CA)

(72) Inventors: Frédéric MANGEAT, Lumbin (FR); Gabriel MANGEAT, Montreal (CA); Marie-Christine MARMETTE, Montreal (CA); Jacques MICHIELS, Montreal (CA)

(21) Appl. No.: 18/568,916

(22) PCT Filed: Jun. 10, 2022
(86) PCT No.: PCT/CA2022/050932

§ 371 Date: Dec. 11, 2023

Related U.S. Application Data
(60) Provisional application No. 63/202,440, filed on Jun. 11, 2021.

(51) Int. Cl. G01N 33/24; G01N 27/22; G01V 3/08

(52) U.S. Cl. CPC G01N 33/24; G01N 27/22; G01V 3/088

(57) ABSTRACT

A probe including a tubular body having a bottom portion, a circuit board mounted within the tubular body and aligned with the bottom portion, a signal generator configured to produce driving signals, each having a frequency included in a range extending from 2 kHz to 200 MHZ, an antenna wrapping an outer surface of the bottom portion of the tubular body, the antenna being coupled with a soil when the probe is inserted in an underground area and configured to produce an electric field upon reception of one of driving signal, the antenna including a ground coil and a signal coil adapted to provide a differential measurement, and a measuring unit configured to determine a capacitance of the soil, based on a collection of differential measurements obtained at different frequencies, the capacitance of the soil being representative of at least one characteristic of the soil.

### TECHNICAL FIELD

The technical field generally relates to systems and methods for measuring soil properties, and more particularly concerns techniques for in situ soil analysis.

### BACKGROUND

Soil tests have been historically performed in a laboratory. Several soil samples are typically collected, which may be achieved by extracting the samples from a field. Once the samples have been extracted, they are sent to the laboratory for subsequent analyses and characterization.

It is known that the characteristics of a soil sample may change or evolve over time. For instance, some characteristics of the extracted samples may be altered during their transport or when they are stored. Thus, the results of the analyses performed on such altered soil samples may not be representative of the actual soil characteristics in situ. The characteristics of the soil also can also vary within the same field. As laboratory characterizations are time consuming and generally expensive, only one laboratory analysis is traditionally performed per field, resulting in a relatively poor characterization of the field. There is thus a need for a system, device, as well as methods that address or alleviate at least some of the challenges presented above.

### SUMMARY

In accordance with one aspect, there is provided a probe for analysing a soil located in an underground area, the probe including:
a tubular body having a bottom portion and a top portion;
a circuit board mounted within the tubular body and being aligned with the bottom portion;
a signal generator operatively connected to the circuit board, the signal generator being configured to produce a plurality of driving signals, each driving signal having a central frequency included in a range extending from about 2 kHz to about 200 MHz;
an antenna wrapping an outer surface of the bottom portion of the tubular body and being operatively connected to the circuit board and to the signal generator, the antenna being electromagnetically coupled with the soil when the probe is inserted in the underground area and being configured to produce an electric field upon reception of one of said plurality of driving signals, the antenna including a ground coil and a signal coil adapted to provide a differential measurement; and
a measuring unit operatively connected to the antenna and being configured to determine a capacitance of the soil, based on a collection of differential measurements obtained at different frequencies, the capacitance of the soil being representative of at least one characteristic of the soil.

In some embodiments, the probe further includes a reference capacitor, and the differential measurement includes:
a first capacitance measurement, the first capacitance measurement being measured between the ground coil and the signal coil;
a second capacitance measurement, the second capacitance measurement being measured between the reference capacitor and the ground coil; and
a third capacitance measurement, the third capacitance measurement being measured between the reference capacitor and the signal coil.

In some embodiments, the reference capacitor has a calibrated capacitance.

In some embodiments, the calibrated capacitance is constant. In some embodiments, the calibrated capacitance is constant over time. In some embodiments, the calibrated capacitance is constant in frequency. In some embodiments, the capacitance of the soil determined by the measuring unit is frequency independent.

In some embodiments, the capacitance of the soil determined by the measuring unit is temperature independent.

In some embodiments, the probe further includes a processor configured to measure a phase change.

In some embodiments, the measuring unit and the processor are integrated to form a single measuring and processing module.

In some embodiments, at least a portion of the tubular body is made of steel.

In some embodiments, the bottom portion is made of an abrasion-resistant material.

In some embodiments, the abrasion-resistant material is plastic.

In some embodiments, the abrasion-resistant material is electrically insulating.

In some embodiments, the probe further includes a casing at least partially covering the bottom portion of the tubular body.

In some embodiments, the antenna is entirely covered by the casing.

In some embodiments, the casing is made from an electrically insulating material.

In some embodiments, the ground coil surrounds at least one of the circuit board and the signal generator. In some embodiments, the circuit board is a printed circuit board.

In some embodiments, said at least one characteristic of the soil is selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In accordance with another aspect, there is provided a method for analysing a soil located in an underground area, the method including:
generating a plurality of driving signals, each driving signal having a central frequency included in a range extending from about 2 kHz to about 200 MHz;
sending each driving signal towards an antenna;
generating an electric field in the soil with the antenna, upon reception of one of said plurality of driving signals; and
determining a capacitance of the soil, based on a collection of differential measurements obtained at different frequencies, the capacitance of the soil being representative of at least one characteristic of the soil.

In some embodiments, each differential measurement includes:
determining a first capacitance between a ground coil of the antenna and a signal coil of the antenna;
determining a second capacitance between a reference capacitor and the ground coil of the antenna; and determining a third capacitance between the reference capacitor and the signal coil of the antenna.

In some embodiments, the reference capacitor has a calibrated capacitance.

In some embodiments, the calibrated capacitance is constant. In some embodiments, the calibrated capacitance is constant over time. In some embodiments, the calibrated capacitance is constant in frequency.

In some embodiments, the method further includes measuring a phase change.

In some embodiments, said at least one characteristic of the soil are selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In accordance with another aspect, there is provided a probe for analysing a soil located in an underground area, the probe including:
a tubular body having a bottom portion and a top portion; and
at least one capacitive sensor, each capacitive sensor being mounted within the tubular body, in the bottom portion, and including:
   a circuit board;
   a signal generator operatively connected to the circuit board, the signal generator being configured to produce a plurality of driving signals, each driving signal having a central frequency included in a range extending from about 2 kHz to about 200 MHz;
   an antenna wrapping an outer surface of the bottom portion of the tubular body and being operatively connected to the circuit board and to the signal generator, the antenna being electromagnetically coupled with the soil when the probe is inserted in the underground area and being configured to produce an electric field upon reception of one of said plurality of driving signals, the antenna including a ground coil and a signal coil adapted to provide a differential measurement; and
   a measuring unit operatively connected to the antenna and being configured to determine a capacitance of the soil, based on a collection of differential measurements obtained at different frequencies, the capacitance of the soil being representative of at least one characteristic of the soil.

In some embodiments, said at least one capacitive sensor is a stack of capacitive sensors.

In some embodiments, two subsequent capacitive sensors of the stack of capacitive sensors are separated by a distance of about 6 inches.

In some embodiments, the probe further includes a reference capacitor, wherein the differential measurement includes:
a first capacitance measurement, the first capacitance measurement being measured between the ground coil and the signal coil;
a second capacitance measurement, the second capacitance measurement being measured between the reference capacitor and the ground coil; and
a third capacitance measurement, the third capacitance measurement being measured between the reference capacitor and the signal coil.

In some embodiments, the reference capacitor has a calibrated capacitance.

In some embodiments, the calibrated capacitance is constant over time and in frequency.

In some embodiments, the capacitance of the soil determined by the measuring unit is frequency independent.

In some embodiments, the capacitance of the soil determined by the measuring unit is temperature independent.

In some embodiments, the probe further includes a processor configured to measure a phase change.

In some embodiments, the measuring unit and the processor are integrated.

In some embodiments, at least a portion of the tubular body is made of steel.

In some embodiments, the bottom portion is made of an abrasion-resistant material.

In some embodiments, the abrasion-resistant material is plastic.

In some embodiments, the abrasion-resistant material is electrically insulating.

In some embodiments, the probe further includes a casing at least partially covering the bottom portion of the tubular body.

In some embodiments, an entire portion of the antenna is covered by the casing.

In some embodiments, the casing is made from an electrically insulating material.

In some embodiments, the ground coil surrounds at least one of the circuit board and the signal generator.

In some embodiments, the circuit board is a printed circuit board.

In some embodiments, said at least one characteristic of the soil are selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In accordance with one aspect, there is provided a probe for analysing a soil, the probe including:
a tubular body;
a circuit board mounted within the tubular body;
a signal generator operatively connected to the circuit board, the signal generator being configured to produce at least one driving signal, each driving signal having a central frequency included in a range extending from about 2 kHz to about 200 MHz;
an antenna wrapping a portion of the tubular body and being operatively connected to the circuit board and to the signal generator, the antenna being electromagnetically coupled with the soil when the probe is inserted therein and being configured to produce an electric field upon reception of said at least one of driving signal, the antenna including a ground coil and a signal coil adapted to provide a differential measurement; and
a measuring unit operatively connected to the antenna and being configured to determine a capacitance of the soil, based on a collection of differential measurements, the capacitance of the soil being representative of at least one characteristic of the soil.

In accordance with one aspect, there is provided an ionic concentration-measuring device for measuring an ionic concentration of a solution, the ionic concentration-measuring device including:
an elongated body insertable in the solution;
a circuit board mounted within the elongated body;
a signal generator operatively connected to the circuit board, the signal generator being configured to produce a plurality of driving signals, each driving signal having a central frequency included in a range extending from about 2 kHz to about 200 MHz;
an antenna wrapped around a portion of the elongated body and being operatively connected to the circuit board and to the signal generator, the antenna being electromagnetically coupled with the solution when the ionic concentration-measuring device is immersed therein and being configured to produce an electromagnetic field upon reception of one of said plurality of driving signals, the antenna including a ground coil and a signal coil adapted to provide a differential measurement; and
a measuring unit operatively connected to the antenna and being configured to determine a capacitance of the solution, based on a collection of differential measurements obtained at different frequencies, the capacitance of the solution being representative of the ionic concentration of the solution.

In some embodiments, the ionic concentration-measuring device further includes a reference capacitor, wherein the differential measurement includes:
a first capacitance measurement, the first capacitance measurement being measured between the ground coil and the signal coil;
a second capacitance measurement, the second capacitance measurement being measured between the reference capacitor and the ground coil; and
a third capacitance measurement, the third capacitance measurement being measured between the reference capacitor and the signal coil.

In some embodiments, the reference capacitor has a calibrated capacitance.

In some embodiments, the calibrated capacitance is constant over time.

In some embodiments, the calibrated capacitance is constant in frequency.

In some embodiments, the ionic concentration-measuring device further includes a processor configured to measure a phase change.

In some embodiments, the measuring unit and the processor are integrated to form a single measuring and processing module.

In some embodiments, at least a portion of the elongated body is made of steel.

In some embodiments, a bottom portion of the elongated body is made of an abrasion-resistant material.

In some embodiments, the abrasion-resistant material is plastic.

In some embodiments, the abrasion-resistant is electrically insulating.

In some embodiments, the ionic concentration-measuring device further includes a casing at least partially covering the bottom portion of the elongated body.

In some embodiments, the antenna is entirely covered by the casing.

In some embodiments, the casing is made from an electrically insulating material.

In some embodiments, the ground coil surrounds at least one of the circuit board and the signal generator.

In some embodiments, the circuit board is a printed circuit board.

In some embodiments, the elongated body is a tubular body.

In accordance with one aspect, there is provided a method for measuring an ionic concentration of a solution, the method including:
generating a plurality of driving signals, each driving signal having a central frequency included in a range extending from about 2 kHz to about 200 MHz;
sending each driving signal towards an antenna;
generating an electric field in the solution with the antenna, upon reception of one of said plurality of driving signals; and
determining a capacitance of the solution, based on a collection of differential measurements obtained at different frequencies, the capacitance of the solution being representative of the ionic concentration of the solution.

In some embodiments, each differential measurement includes:
determining a first capacitance between a ground coil of the antenna and a signal coil of the antenna;
determining a second capacitance between a reference capacitor and the ground coil of the antenna; and
determining a third capacitance between the reference capacitor and the signal coil of the antenna.

In some embodiments, the reference capacitor has a calibrated capacitance.

In some embodiments, the calibrated capacitance is constant over time.

In some embodiments, the calibrated capacitance is constant in frequency.

In some embodiments, the method further includes measuring a phase change.

Other features and advantages of the present description will become more apparent upon reading of the following non-restrictive description of specific embodiments thereof, given by way of example only with reference to the accompanying drawings.

### BRIEF DESCRIPTION OF THE DRAWINGS

FIG. 1 is a representation of a signal generator and a measuring unit of a probe for analysing a soil located in an underground area, in accordance with one embodiment.

FIGS. 2A-D schematically illustrate a bottom portion of the probe as disclosed herein, wherein an antenna is mounted around the bottom portion of the probe, the antenna generating an electric field in the soil.

FIGS. 3A-B illustrate experimental results obtained with the probe as disclosed herein.

FIG. 4A illustrates a perspective view of a portion of the probe, in accordance with one embodiment.

FIG. 4B illustrates a portion of the probe, in accordance with one embodiment.

FIG. 5 presents a plurality of measurement signals produced with an embodiment of the probe having been herein described. Each of the measurement signals was obtained in different soil textures at 24.5% humidity (volume/volume).

FIG. 6 presents a plurality of measurement signals produced with an embodiment of the probe having been herein described. Each of the measurement signals was obtained in solutions with 100 ppm of different elements.

FIG. 7 presents a plurality of measurement signals produced with an embodiment of the probe having been herein described. Each of the measurement signals was obtained in sandy clay loam soil at a different soil humidity (volume/volume).

FIG. 8 presents a plurality of measurement signals produced with an embodiment of the probe having been herein described. Each of the measurement signals was obtained in a plurality of nutrient solutions, each having a different ionic concentration of the same compound (NO₃).

### DETAILED DESCRIPTION

In the following description, similar features in the drawings have been given similar reference numerals, and, to not unduly encumber the figures, some elements may not be indicated on some figures if they were already identified in one or more preceding figures. It should also be understood herein that the elements of the drawings are not necessarily depicted to scale, since emphasis is placed upon clearly illustrating the elements and structures of the present embodiments.

The terms “a”, “an” and “one” are defined herein to mean “at least one”, that is, these terms do not exclude a plural number of elements, unless stated otherwise. It should also be noted that terms such as “substantially”, “generally” and “about”, that modify a value, condition, or characteristic of a feature of an exemplary embodiment, should be understood to mean that the value, condition or characteristic is defined within tolerances that are acceptable for the proper operation of this exemplary embodiment for its intended application.

In the present description, the terms “connected”, “coupled”, and variants and derivatives thereof, refer to any connection or coupling, either direct or indirect, between two or more elements. The connection or coupling between the elements may be acoustical, mechanical, physical, optical, operational, electrical, wireless, or a combination thereof.

In the present description, the expression “based on” is intended to mean “based at least partly on”, that is, this expression can mean “based solely on” or “based partially on”, and so should not be interpreted in a limited manner. More particularly, the expression “based on” could also be understood as meaning “depending on”, “representative of”, “indicative of”, “associated with” or similar expressions.

It will be appreciated that positional descriptors indicating the position or orientation of one element with respect to another element are used herein for ease and clarity of description and should, unless otherwise indicated, be taken in the context of the figures and should not be considered limiting. It will be understood that spatially relative terms (e.g., “outer” and “inner”, “outside” and “inside” and “top” and “bottom”) are intended to encompass different positions and orientations in use or operation of the present embodiments, in addition to the positions and orientations exemplified in the figures.

The term “field” is herein used to refer to a region of land where trees, plants, crops and the like usually grow. The term “soil” is herein used for qualifying the underground area beneath the surface of the field, which may include the surface or a portion thereof. It should be noted that the expressions “trees”, “plants”, “crops”, synonyms and derivatives thereof may encompass a broad variety of organisms and should not be considered limitative. Nonlimitative examples of trees, plants or crops may include seedlings, ornamental crops, ornamental plants, plugs, liners, fruits, small fruits, vegetables, leafy greens, herbs, young plants, high-value crops, perennial plants, annual plants, biennial plants, grain, grass, cereal, and many others. The trees, plants or crops may be produced for human food, non-human food, or non-food applications. Of note, the present techniques may be used to characterize different substrates such as, for example and without being limitative: compost, manure, food, and/or plants. Of course, these examples are nonlimitative and serve an illustrative purpose only.

Broadly described, there is provided a probe for analysing a soil located in an underground area of a field. The probe may be an optical probe, and so may rely on spectroscopy. More specifically, there is provided a capacitive sensor for such a probe or optical probe, in order to evaluate electric or electromagnetic properties of the soil. The capacitive sensor is configured to determine or measure a capacitance of the soil, based on a collection of differential measurements obtained at different frequencies. The capacitance of the soil is representative of at least one characteristic of the soil, and so determining or measuring the capacitance of the soil may contribute to the characterization of the field being analysed.

The capacitive sensor allows assessing in real time, or near real time, different electric and/or electromagnetic characteristics of the soil, which are a subset of properties of what may be referred to as the global soil condition. The soil condition may include many other properties than the electric and/or electromagnetic characteristics of the soil, as it will be explained in greater detail later.

The embodiments of the probe that will be herein described broadly rely on capacitance measurements and the like, and more specifically on the evaluation of an interaction between an electric field generated by the probe (i.e., the capacitive sensor) for determining the electric and/or electromagnetic characteristics of the soil.

The probe can be inserted in the underground area of a field to measure and monitor the soil condition in situ, i.e., without the need to extract a soil sample from the field prior to its characterization, thereby providing a dynamic characterization of the soil, instead of a single static measurement of the soil condition, which is typically obtained in a laboratory. In some embodiments, the probe can be sequentially moved from one location to another to take measurements at different locations of the field being characterized, thereby allowing to obtain a global representation or a cartography of the field. In some embodiments, a plurality of optical probes may be installed in the field, and the cartography of the field may be obtained by combining the measurements and results collected with each probe.

In some embodiments, the dynamic characterization of the soil may be used to plan the maintenance of the field, plan the fertilization of the field, evaluate, and potentially prevent the risk of diseases for the tree(s), plant(s) and/or crop(s) growing in the field, and the like.

Examples of optical probes compatible with the technology being herein described are presented in PCT/CA2019/051322 and PCT/CA2021/050233, which content is incorporated herein by reference.

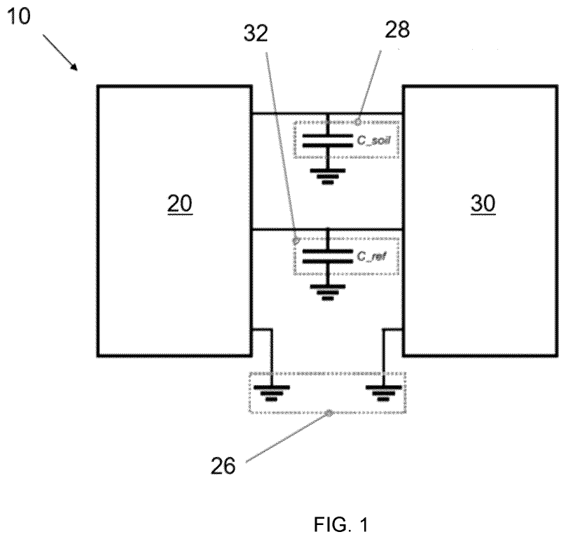

Now turning to FIGS. 1 to 8, various embodiments of the probe 10 will be described. The probe 10 includes an elongated body, and preferably a tubular body 12 having a bottom portion 14 and a top portion 16. The probe 10 also includes a capacitive sensor, the capacitive sensor including at least a circuit board 18, a signal generator 20, and an antenna 22. The probe 10 also includes a measuring unit 30. The measuring unit 30 may be integrated to the capacitive sensor or may alternatively be provided as a separate unit operatively connected to the capacitive sensor.

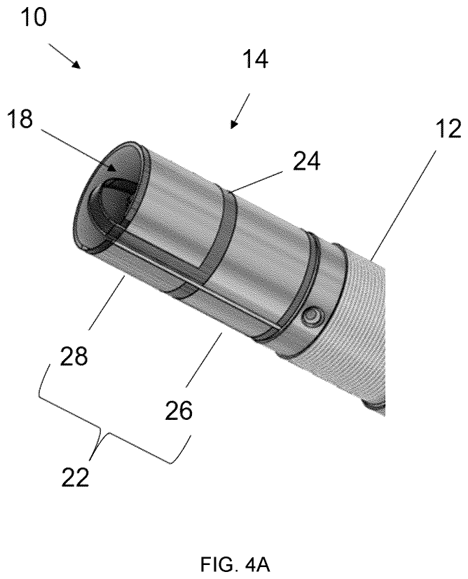

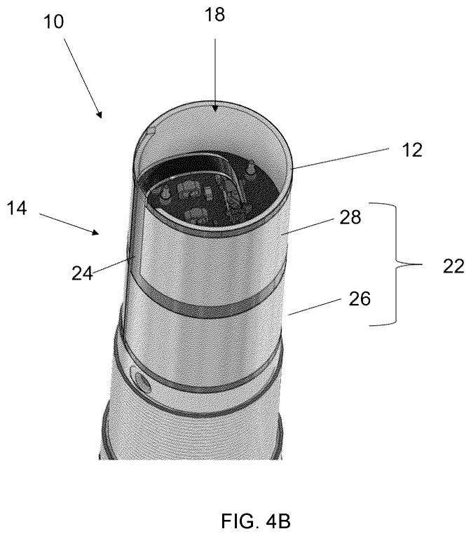

The circuit board 18 is mounted within the tubular body 12, as illustrated for example in FIGS. 4A-B. The circuit board 18 is substantially aligned with the bottom portion 14, meaning that the circuit board 18 is provided in the bottom portion 14 of the tubular body 12. In some embodiments, the circuit board 18 is a printed circuit board 18. Of note, the bottom portion 14 refers to the portion of the tubular body 12 that is exposed to the underground area when the probe 10 is inserted into the ground.

It should be noted that the tubular body 12 may be made from a material impermeable to the soil solution present in the soil, i.e., the soil solution cannot diffuse or circulate within the tubular body 12 (or portion(s) thereof), and so does not penetrate the tubular body 12. As such, the probe 10, and more specifically the tubular body 12 is generally made from a non-porous material, or the porosity of the material is such that the soil solution stays outside of the tubular body 12.

The signal generator 20 is operatively connected to the circuit board 18 and is configured to produce a plurality of driving signals (collectively referred to as the “driving signals”). Each driving signal has a corresponding central frequency and the driving signals each have a different central frequency one from another. The signal generator 20 is configured to generate driving signals over a relatively wide range of frequencies. The driving signals may be referred to as broadband driving signals or multifrequency driving signals. The central frequency of the driving signals is included in a range extending from about 2 kHz to about 200 MHz.

In some embodiments, the signal generator 20 is configured for generating driving signals in a continuous regime. It will however be readily understood that in other embodiments, the signal generator 20 could be operated either in a continuous regime or an intermittent regime, according to one's needs and/or the targeted application(s). One skilled in the art will readily understand that the choice and the configuration of the signal generator 20 may be limited and/or influenced by the predetermined parameters dictated by a given application.

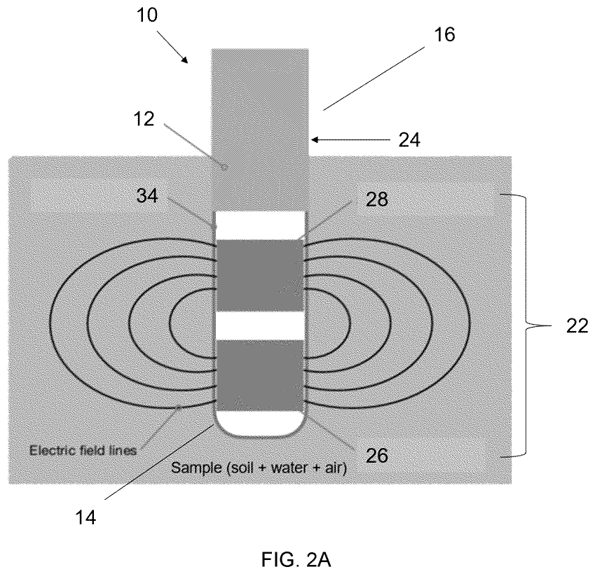

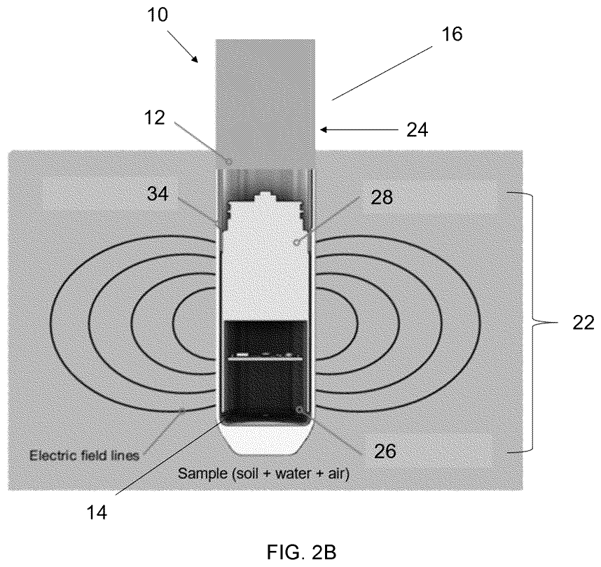

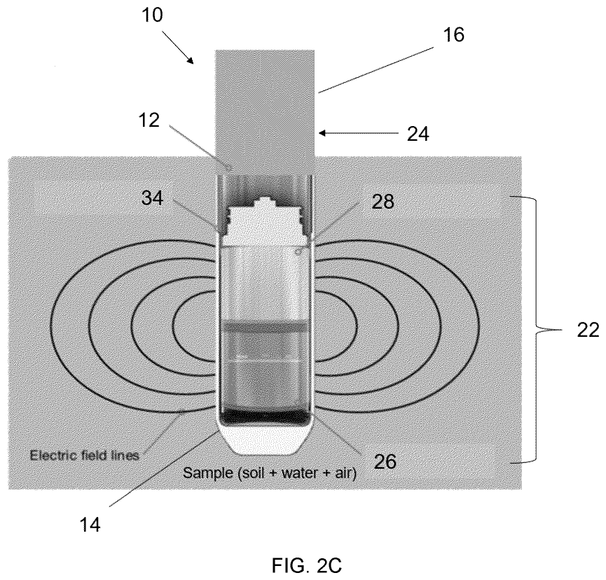

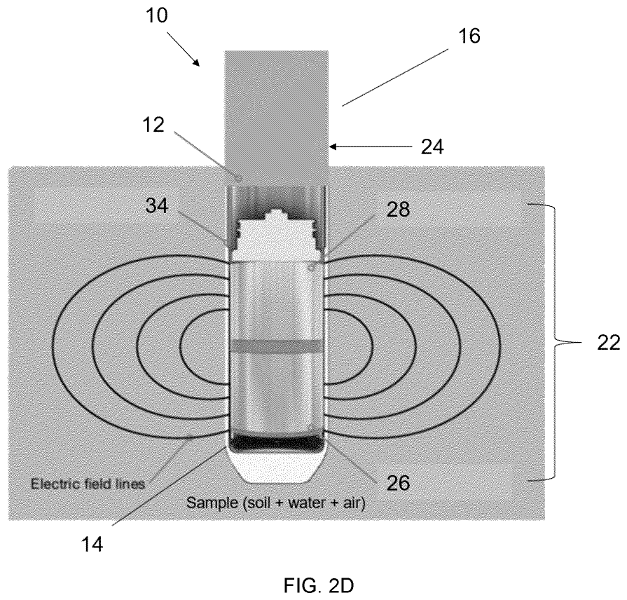

The antenna 22 wraps or at least partially surrounds an outer surface 24 of the bottom portion 14 of the tubular body 12, as better illustrated in FIGS. 4A-B. The antenna 22 is operatively connected to the circuit board 18 and to the signal generator 20. When the probe 10 is inserted in the underground area, the antenna 22 is electrically and/or electromagnetically coupled with the soil. The antenna 22 is configured to produce or generate an electric field in the soil, upon reception of one of the driving signals. Now turning to FIGS. 2A-D, the antenna 22 includes a ground coil 28 and a signal coil 26 (sometimes referred to as the “antenna coil”). The ground coil 28 and the signal coil 26 are adapted to provide a differential measurement. In some embodiments, the antenna 22 may be a multipole antenna. In some embodiments, the antenna 22 may be a multipattern antenna.

In some embodiments, the ground coil 28 of the antenna 22 surrounds at least one of the circuit board 18 and the signal generator 20. This configuration may be useful to minimise or at least reduce the generation of parasite signals within the probe 10 or capacitive sensor.

It should be noted that the signal generator 20 and the measuring unit 30 are relatively close to the antenna 22. One benefit of this configuration is that the probe 10 does not need or require coaxial cables and/or shielding components to carry the signal without distortion. In some embodiments, the ground coil 28 of the antenna 22 is separated from a surface of the circuit board 18 by a distance of about 1 cm. In some embodiments, the ground coil 28 is connected to the circuit board 18 using an appropriate connecting mechanism, the connecting mechanism having a length of less than about 5 cm.

The electrical configuration and design of the probe 10 is relatively compact and cost-effective with respect to available commercial solutions. In addition, the electrical components of the capacitive sensor are relatively close one with respect to another, and so are generally maintained at the same temperature, therefore mitigating, or at least reducing potential thermal effects that would negatively affect the precision or reliability of the differential measurements. The differential measurements are also generally not affected by the nonlinearities of some of the components of the capacitive sensor. More specifically, the nonlinearities of these components encompass at least one of the following: temperature-related non-linearity, voltage-related non-linearity and frequency-related non-linearity. In fact, each component has a voltage response which may linearly or non-linearly depends on temperature and/or frequency. The differential measurement may either cancel out or at least reduce this dependence. The outcome of the differential measurements is therefore representative of the capacitance of the soil and/or a variation in the capacitance of the soil.

In some embodiments, the probe 10 further includes a reference capacitor 32. The differential measurement may include three capacitance measurements, respectively referred to as a first capacitance measurement, a second capacitance measurement and a third capacitance measurement. The first capacitance measurement may be measured between the ground coil 28 and the signal coil 26. The second capacitance measurement may be measured between the reference capacitor 32 and the ground coil 28. The third capacitance measurement may be measured between the reference capacitor 32 and the signal coil 26.

In some embodiments, the reference capacitor 32 has a calibrated capacitance. In some embodiments, the calibrated capacitance is constant.

The measuring unit 30 is operatively connected to the antenna 22 and is configured to determine a capacitance of the soil. The determination of the capacitance of the soil is based on a collection of differential measurements obtained at different frequencies. As the capacitance is representative of an interaction between the electric field produced by the antenna 22 and the soil, the capacitance of the soil is representative of at least one characteristic of the soil.

In some embodiments, the capacitance of the soil may be normalized with respect to the capacitance of air. Doing so allows obtaining the permittivity of the soil as a function of the frequency.

In some embodiments, the capacitance of the soil determined by the measuring unit 30 is frequency independent and/or temperature independent.

In some embodiments, the probe 10 further includes a processor configured to measure a phase change. Combining the phase change and the permittivity of the soil allows obtaining the real and imaginary components of the permittivity, which may provide insight on the conductivity of the soil. In some embodiments, the measuring unit 30 and the processor are integrated. In some embodiments, the processor may be or may include an external computer. The external computer can be operatively connected to the probe 10, either wirelessly or through physical connection. The term “computer” (or “computing device”) is used to encompass computers, servers and/or specialized electronic devices which receive, process and/or transmit data. Computers are generally part of “systems” and include processing means, such as microcontrollers and/or microprocessors, CPUs or are implemented on FPGAs, as examples only. The processing means are used in combination with storage medium, also referred to as “memory” or “storage means”. Storage medium can store instructions, algorithms, rules and/or data to be processed. Storage medium encompasses volatile or non-volatile/persistent memory, such as registers, cache, RAM, flash memory, ROM, as examples only. The type of memory is, of course, chosen according to the desired use, whether it should retain instructions, or temporarily store, retain or update data. One skilled in the art will therefore understand that each such computer typically includes a processor (or multiple processors) that executes program instructions stored in the memory or other non-transitory computer-readable storage medium or device (e.g., solid state storage devices, disk drives, and the like). The various functions, modules, services, units or the like disclosed hereinbelow can be embodied in such program instructions, and/or can be implemented in application-specific circuitry (e.g., ASICs or FPGAs) of the computers. Where a computer system includes multiple computers, these devices can, but need not, be co-located. In some embodiments, a computer system can be a cloud-based computing system whose processing resources are shared by multiple distinct business entities or other users. As it will be readily understood, the processor can be implemented as a single unit or as a plurality of interconnected processing sub-units. Also, the processor can be embodied by a computer, a microprocessor, a microcontroller, a central processing unit, or by any other type of processing resource or any combination of such processing resources configured to operate collectively as a processor. The processor can be implemented in hardware, software, firmware, or any combination thereof, and be connected to the various components of the spectral identification system via appropriate communication ports.

In some embodiments, at least a portion of the tubular body 12 is made of steel. In some embodiments, the bottom portion 14 is made of an abrasion-resistant material. In some embodiments, the abrasion-resistant material is plastic. In some embodiments, the abrasion-resistant material is electrically insulating.

In some embodiments, the probe 10 further includes a casing 34 at least partially covering the bottom portion 14 of the tubular body 12. In some embodiments, an entire portion of the antenna 22 is covered by the casing 34. The casing 34 may be made from a broad variety of material but is preferably made from an electrically insulating material. For example, and without being limitative, the casing 34 may be made from polymers, such as, to name a few, vinyl, fiberglass and rigid polyvinyl chloride (PVC), or any other electrically insulating material(s) or combination(s) of electrically insulating materials, or any other material that can be used to house and, in some instances, protect the bottom portion 14 of the tubular body 12 and/or the antenna 22. The casing 34 may have any geometrical configurations (i.e., size and dimensions). The casing 34 may have a substantially cylindrical shape. At least one end of the casing 34 may be opened, and another end, for example the end opposite the opened end, may be closed.

The probe 10 typically includes at least one capacitive sensor. In some embodiments, the probe 10 may include a plurality of capacitive sensors. As explained above, a capacitive sensor according to the present technology includes at least a circuit board 18, a signal generator 20 and an antenna 22. In some embodiments, the plurality of capacitive sensors may be embodied by a stack of capacitive sensors. In some embodiments, two subsequent capacitive sensors of the stack of capacitive sensors may be separated by a distance of about 6 inches. In some embodiments, each capacitive sensor is equipped with a dedicated measuring unit 30.

In some embodiments, the characteristics of the soil are selected from the group consisting of permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In some embodiments, the probe 10 further includes a stopper mechanically engageable with an outer periphery of the tubular body 12. The stopper may be engaged with the outer periphery of the tubular body 12 at an adjustable height (i.e., with respect with a longitudinal axis of the tubular body 12). When the stopper is mechanically engaged with the outer periphery of the tubular body 12, the stopper outwardly and radially extends from the outer periphery of the tubular. During the measurements, the probe 10 is inserted in the underground area of the soil and the stopper abuts the surface of the soil to prevent a deeper insertion of the probe 10 in the underground area of the soil. One benefit associated with the stopper is that when it is mechanically engaged at a predetermined height of the tubular body 12, it is possible to perform measurement at a predetermined depth in the soil, which may help in obtaining more reliable and precise measurements. In some embodiments, the stopper may be slidably engaged with the tubular body 12 of the probe 10, and the height of the stopper (with respect to the tubular body 12) may be adjusted by sliding the stopper along a longitudinal direction parallel with the longitudinal axis of the tubular body 12. Of note, the stopper may be formed from one component or may alternatively include a plurality of components.

In some embodiments, the tubular body 12 of the probe 10, or at least a portion thereof, may be graduated or provided with a label representative of various geometric parameters such as, for example, a dimension of the tubular body 12. For example, and without being limitative, the tubular body 12 may be marked along its longitudinal axis to indicate the depth at which the probe 10 is inserted with respect to a dimension of the probe 10 or a component thereof.

In some embodiments, the probe 10 may include the graduated tubular body 12 and the stopper as described above and may therefore be configured to provide measurements at a predetermined depth (which may be adjusted) in the underground area of the soil. In some embodiments, the predetermined depth may be 6 inches, 12 inches, 18 inches or 24 inches.

In some embodiments, the probe 10 is configured to perform measurements of the characteristics of the soil near the surface of the soil, close to the roots of a plant or crop and even beneath the roots of the plant or crop.

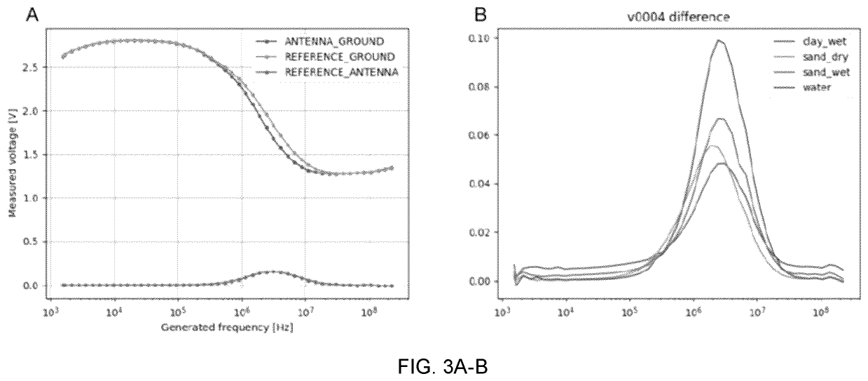

Now turning to FIGS. 3A-B, some experimental data that having been obtained with the probe 10 having been herein described will be presented.

FIG. 3A illustrates raw measurement made between the signal (antenna 22) coil and the ground coil 28, the reference capacitor 32 and the ground coil 28, and the reference capacitor 32 and the signal (antenna 22) coil. It should be noted that the measurement of the reference capacitance generally varies with the frequency (i.e., is frequency dependent), which is due to the frequency dependency of the whole system (i.e., the probe 10). The differential measurements allows minimising or at least significantly reducing the contribution or negative impacts of several confounding factors, such as, for example, temperature and frequency.

FIG. 3B illustrates raw measurement made in different media: clay, a dry soil, a wet soil and water. The probe 10 having been herein described allows measuring the texture of the soil (percentage of clay, loam and silt), the water content of the, and/or the organic matter content of the soil.

In accordance with another aspect, there is provided a method for analysing a soil located in an underground area.

The method includes a first general step of generating a plurality of driving signals. As previously explained, each driving signal has a central frequency included in a range extending from about 2 kHz to about 200 MHz.

The method also includes a step of sending each driving signal towards an antenna 22.

Following the step of sending the driving signals to the antenna 22, the method includes a step of generating an electric field in the soil with the antenna 22. This step is carried out upon reception of one or more of the driving signals.

The method then includes a step of determining a capacitance of the soil, based on a collection of differential measurements obtained at different frequencies. As explained above, the capacitance of the soil is representative of at least one characteristic of the soil.

In some embodiments, each differential measurement may include three sub-steps, namely determining a first capacitance between a ground coil 28 of the antenna 22 and a signal coil 26 of the antenna 22, determining a second capacitance between a reference capacitor 32 and the ground coil 28 of the antenna 22 and determining a third capacitance between the reference capacitor 32 and the signal coil 26 of the antenna 22.

In some embodiments, the reference capacitor 32 may have a calibrated capacitance. The calibrated capacitance may be constant.

In some embodiments, the method further includes measuring a phase change.

In some embodiments, said at least one characteristic of the soil are selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In accordance with another aspect, there is provided a probe for analysing a soil located in an underground area. The probe includes a tubular body having a bottom portion and a top portion. The probe includes at least one capacitive sensor. Each capacitive sensor is mounted within the tubular body, in the bottom portion. Each capacitive sensor includes a circuit board, a signal generator, an antenna and a measuring unit. The signal generator is operatively connected to the circuit board. The signal generator is configured to produce a plurality of driving signals, each driving signal having a central frequency included in a range extending from about 2 kHz to about 200 MHz. The antenna wraps an outer surface of the bottom portion of the tubular body and is operatively connected to the circuit board and to the signal generator. The antenna is electromagnetically coupled with the soil when the probe is inserted in the underground area and is configured to produce an electric field upon reception of one of said plurality of driving signals. The antenna includes a ground coil and a signal coil adapted to provide a differential measurement. The measuring unit is operatively connected to the antenna and is configured to determine a capacitance of the soil, based on a collection of differential measurements obtained at different frequencies. The capacitance of the soil being representative of at least one characteristic of the soil.

In some embodiments, said at least one capacitive sensor is a stack of capacitive sensors.

In some embodiments, two subsequent capacitive sensors of the stack of capacitive sensors are separated by a distance of about 6 inches.

In some embodiments, the probe further includes a reference capacitor, wherein the differential measurement includes a first capacitance measurement, the first capacitance measurement being measured between the ground coil and the signal coil: a second capacitance measurement, the second capacitance measurement being measured between the reference capacitor and the ground coil; and a third capacitance measurement, the third capacitance measurement being measured between the reference capacitor and the signal coil.

In some embodiments, the reference capacitor has a calibrated capacitance. In some embodiments, the calibrated capacitance is constant over time and in frequency. In some embodiments, the capacitance of the soil determined by the measuring unit is frequency independent.

In some embodiments, the capacitance of the soil determined by the measuring unit is temperature independent.

In some embodiments, the probe further includes a processor configured to measure a phase change. In some embodiments, the measuring unit and the processor are integrated.

In some embodiments, at least a portion of the tubular body is made of steel. In some embodiments, the bottom portion is made of an abrasion-resistant material. In some embodiments, the abrasion-resistant material is plastic. In some embodiments, the abrasion-resistant material is electrically insulating.

In some embodiments, the probe further includes a casing at least partially covering the bottom portion of the tubular body. In some embodiments, an entire portion of the antenna is covered by the casing. In some embodiments, the casing is made from an electrically insulating material. In some embodiments, the ground coil surrounds at least one of the circuit board and the signal generator.

In some embodiments, the circuit board is a printed circuit board.

In some embodiments, said at least one characteristic of the soil are selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of the soil solution, temperature, moisture, soil water content, soil water potential and pH.

In accordance with one aspect, there is provided a probe for analysing a soil, the probe includes a tubular body: a circuit board mounted within the tubular body: a signal generator operatively connected to the circuit board, the signal generator being configured to produce at least one driving signal, each driving signal having a central frequency included in a range extending from about 2 kHz to about 200 MHz: an antenna wrapping a portion of the tubular body and being operatively connected to the circuit board and to the signal generator, the antenna being electromagnetically coupled with the soil when the probe is inserted therein and being configured to produce an electric field upon reception of said at least one of driving signal, the antenna including a ground coil and a signal coil adapted to provide a differential measurement; and a measuring unit operatively connected to the antenna and being configured to determine a capacitance of the soil, based on a collection of differential measurements, the capacitance of the soil being representative of at least one characteristic of the soil.

### Ionic Concentration Measurements

In accordance with one aspect, there is provided an ionic concentration-measuring device for measuring an ionic concentration of a solution. The ionic concentration-measuring device includes an elongated body insertable in the solution, a circuit board, a signal generator, an antenna, and a measuring unit. The circuit board is mounted within the elongated body. The signal generator is operatively connected to the circuit board. The signal generator is configured to produce a plurality of driving signals, each driving signal having a central frequency included in a range extending from about 2 kHz to about 200 MHz. The antenna is wrapped around a portion of the elongated body and is operatively connected to the circuit board and to the signal generator. The antenna is electromagnetically coupled with the solution when the ionic concentration-measuring device is immersed therein and is configured to produce an electromagnetic field upon reception of one of said plurality of driving signals. The antenna includes a ground coil and a signal coil adapted to provide a differential measurement. The measuring unit is operatively connected to the antenna and is configured to determine a capacitance of the solution, based on a collection of differential measurements obtained at different frequencies. The capacitance of the solution is representative of the ionic concentration of the solution.

The ionic concentration-measuring device can be used to measure, monitor and/or track concentration of ion(s) in solutions that may be used to irrigate, fertilized, and/or clean plants or crops during their growing process within a horticultural structure. Horticultural structures provide regulated climatic conditions to the plants or crops to facilitate, control, assist and/or accelerate their growth. Nonlimitative examples of horticultural structures include greenhouse, glasshouse, and hothouse.

In some embodiments, the ionic concentration-measuring device can be used in outdoor applications. For example, the ionic-measuring device may be immersed in tanks (or similar receptacle) adapted to receive water or solution that can be used for irrigating and/or fertilizing a soil, e.g., in a field. Of note, the water or solution contained in the tank generally includes chemical elements, compounds, ions, nutrients, and any combinations thereof, and the ionic concentration-measuring device can be adapted to measure a concentration of at least one chemical element, compound, ion, nutrient, and/or any combinations thereof, in the water or solution. In some embodiments, the ionic concentration-measuring device may be coupled or mounted to a mechanical component connected to the tank. Nonlimitative examples of such a mechanical component include a pipe, a tubing, an inlet, an outlet and many others.

In some embodiments, the ionic concentration-measuring device further includes a reference capacitor. The differential measurement includes a first capacitance measurement, the first capacitance measurement being measured between the ground coil and the signal coil: a second capacitance measurement, the second capacitance measurement being measured between the reference capacitor and the ground coil; and a third capacitance measurement, the third capacitance measurement being measured between the reference capacitor and the signal coil.

In some embodiments, the reference capacitor has a calibrated capacitance. In some embodiments, the calibrated capacitance is constant over time. In some embodiments, the calibrated capacitance is constant in frequency.

In some embodiments, the ionic concentration-measuring device further includes a processor configured to measure a phase change.

In some embodiments, the measuring unit and the processor are integrated to form a single measuring and processing module.

In some embodiments, at least a portion of the elongated body is made of steel. In some embodiments, a bottom portion of the elongated body is made of an abrasion-resistant material. In some embodiments, the abrasion-resistant material is plastic. In some embodiments, the abrasion-resistant is electrically insulating. In some embodiments, the ionic concentration-measuring device further includes a casing at least partially covering the bottom portion of the elongated body. In some embodiments, the antenna is entirely covered by the casing. In some embodiments, the casing is made from an electrically insulating material.

In some embodiments, the ground coil surrounds at least one of the circuit board and the signal generator.

In some embodiments, the circuit board is a printed circuit board.

In some embodiments, the elongated body is a tubular body.

In accordance with one aspect, there is provided a method for measuring an ionic concentration of a solution. The method includes generating a plurality of driving signals, each driving signal having a central frequency included in a range extending from about 2 kHz to about 200 MHz. The method includes sending each driving signal towards an antenna. The method includes generating an electric field in the solution with the antenna, upon reception of one of said plurality of driving signals. The method includes determining a capacitance of the solution, based on a collection of differential measurements obtained at different frequencies, the capacitance of the solution being representative of the ionic concentration of the solution.

In some embodiments, each differential measurement includes determining a first capacitance between a ground coil of the antenna and a signal coil of the antenna: determining a second capacitance between a reference capacitor and the ground coil of the antenna; and determining a third capacitance between the reference capacitor and the signal coil of the antenna.

In some embodiments, the reference capacitor has a calibrated capacitance. In some embodiments, the calibrated capacitance is constant over time. In some embodiments, the calibrated capacitance is constant in frequency.

In some embodiments, the method further includes measuring a phase change.

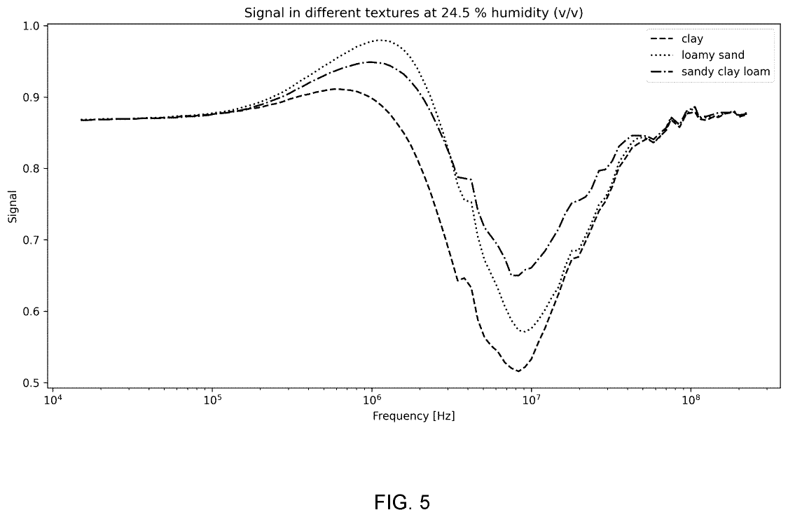

Now turning to FIGS. 5 to 8, other experimental data obtained with the techniques having been herein described will be presented.

FIG. 5 presents a plurality of detection or measurement signals produced with an embodiment of the probe or device having been herein described. Each of the measurement signals was obtained in different soil textures at 24.5% humidity (volume/volume). As illustrated, the data collected with the techniques according to the current disclosure allows distinguishing between several soil textures, such as clay, loamy sand, and sandy clay loam, as a function of the frequency. It will have been readily understood that the techniques may be used in a broad variety of textures.

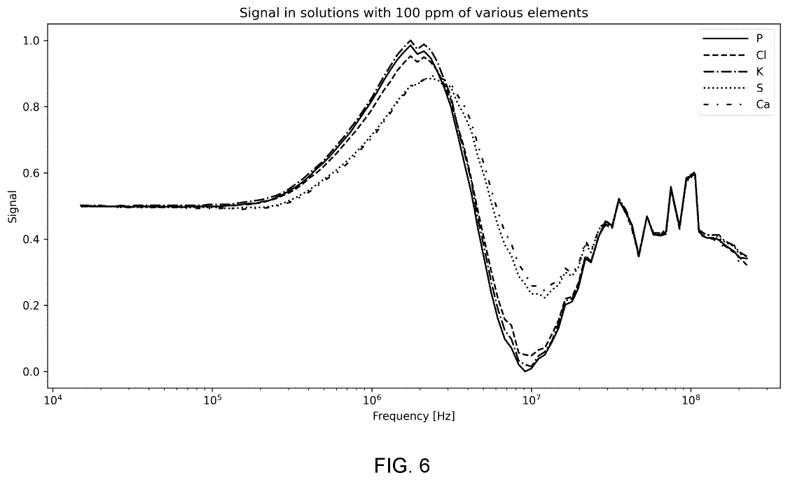

FIG. 6 presents a plurality of measurement signals produced with an embodiment of the probe or device having been herein described. Each of the measurement signals was obtained in solutions with 100 ppm of different elements. As illustrated, the data collected with the techniques according to the current disclosure allows distinguishing between several elements, such as phosphorus, chlore, potassium, sodium and calcium, as a function of the frequency. It will have been readily understood that the techniques may be used to characterized other chemical element(s), ion(s), molecule(s), compound(s) and combination(s) thereof.

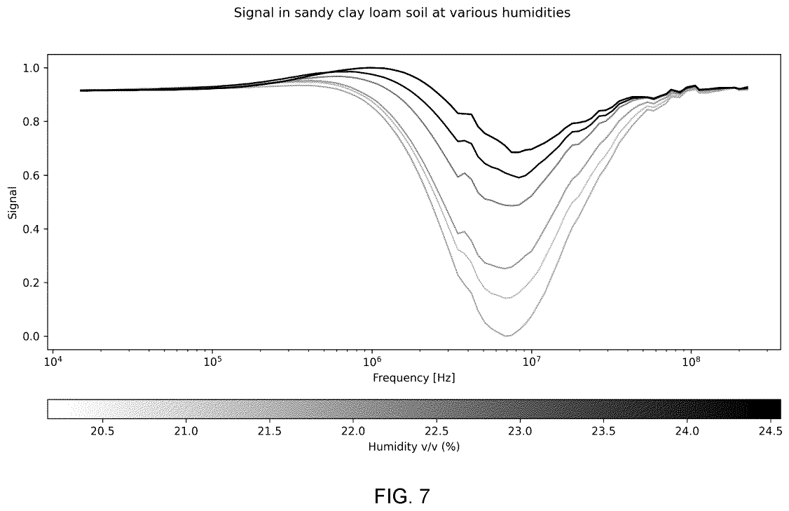

FIG. 7 presents a plurality of measurement signals produced with an embodiment of the probe or device having been herein described. Each of the measurement signals was obtained in sandy clay loam soil at a different soil humidity (volume/volume). As illustrated, the data collected with the techniques according to the current disclosure allows observing the effect of the soil humidity on the signal collected by the probe or device, as a function of the frequency.

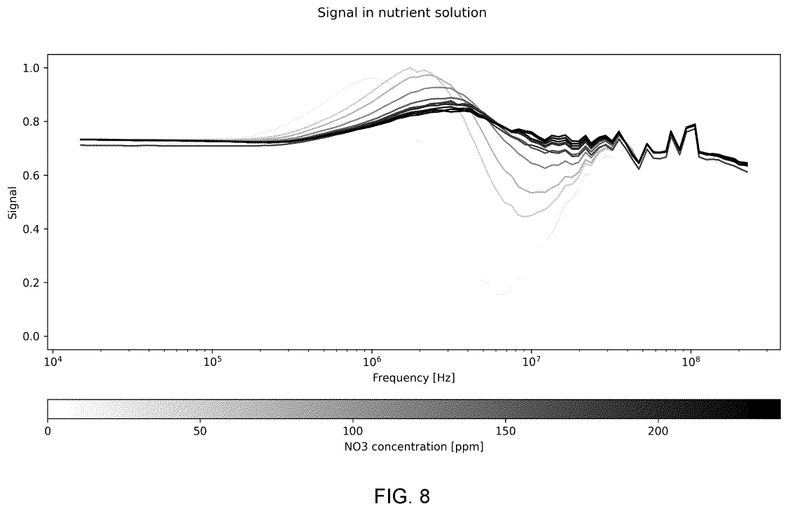

FIG. 8 presents a plurality of measurement signals produced with an embodiment of the probe or device having been herein described. Each of the measurement signals was obtained in a plurality of nutrient solutions, each having a different ionic concentration of the same compound. As illustrated, the data collected with the techniques according to the current disclosure allows observing the effect of the solution concentration on the signal collected by the probe or device, as a function of the frequency. In FIG. 8, the sample being characterized includes NO₃. It should be noted that the concentration of other chemical, element(s), ion(s), compound(s) and/or molecule(s) may also be measured or characterized using the techniques being herein described.

Several alternative embodiments and examples have been described and illustrated herein. The embodiments described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. The present examples and embodiments, therefore, are to be considered in all respects as illustrative and not restrictive. Accordingly, while specific embodiments have been illustrated and described, numerous modifications come to mind without significantly departing from the scope defined in the appended claims.

1-71. (canceled)

72. A probe for analysing a soil located in an underground area, the probe comprising:
a tubular body having a bottom portion and a top portion;
a circuit board mounted within the tubular body and being aligned with the bottom portion;
a signal generator operatively connected to the circuit board, the signal generator being configured to produce a plurality of driving signals, each driving signal having a central frequency comprised in a range extending from about 2 kHz to about 200 MHz;
an antenna wrapping an outer surface of the bottom portion of the tubular body and being operatively connected to the circuit board and to the signal generator, the antenna being electromagnetically coupled with the soil when the probe is inserted in the underground area and being configured to produce an electric field upon reception of one of said plurality of driving signals, the antenna comprising a ground coil and a signal coil adapted to provide a differential measurement; and
a measuring unit operatively connected to the antenna and being configured to determine a capacitance of the soil, based on a collection of differential measurements obtained at different frequencies, the capacitance of the soil being representative of at least one characteristic of the soil.

73. The probe of claim 72, further comprising a reference capacitor, wherein the differential measurement comprises:
a first capacitance measurement, the first capacitance measurement being measured between the ground coil and the signal coil;
a second capacitance measurement, the second capacitance measurement being measured between the reference capacitor and the ground coil; and
a third capacitance measurement, the third capacitance measurement being measured between the reference capacitor and the signal coil.

74. The probe of claim 73, wherein the reference capacitor has a calibrated capacitance.

75. The probe of claim 74, wherein the calibrated capacitance is constant over time.

76. The probe of claim 74, wherein the calibrated capacitance is constant in frequency.

77. The probe of claim 72, wherein the capacitance of the soil determined by the measuring unit is frequency independent.

78. The probe of claim 72, wherein the capacitance of the soil determined by the measuring unit is temperature independent.

79. The probe of claim 72, further comprising a processor configured to measure a phase change.

80. The probe of claim 79, wherein the measuring unit and the processor are integrated to form a single measuring and processing module.

81. The probe of claim 72, further comprising a casing at least partially covering the bottom portion of the tubular body.

82. The probe of claim 72, wherein the ground coil surrounds at least one of the circuit board and the signal generator.

83. The probe of claim 72, wherein said at least one characteristic of the soil is selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of soil solution, temperature, moisture, soil water content, soil water potential and pH.

84. A method for analysing a soil located in an underground area, the method comprising:
generating a plurality of driving signals, each driving signal having a central frequency comprised in a range extending from about 2 kHz to about 200 MHZ;
sending each driving signal towards an antenna;
generating an electric field in the soil with the antenna, upon reception of one of said plurality of driving signals; and
determining a capacitance of the soil, based on a collection of differential measurements obtained at different frequencies, the capacitance of the soil being representative of at least one characteristic of the soil.

85. The method of claim 84, wherein each differential measurement comprises:
determining a first capacitance between a ground coil of the antenna and a signal coil of the antenna;
determining a second capacitance between a reference capacitor and the ground coil of the antenna; and
determining a third capacitance between the reference capacitor and the signal coil of the antenna.

86. The method of claim 84, wherein said at least one characteristic of the soil are selected from the group consisting of: permittivity, soil texture, clay content, loam content, sand content, bulk density, cation exchange capacity (CEC), soil organic matter (SOM), soil organic carbon (SOC), level of nutrients, level of available nutrients, ionic concentration of soil solution, temperature, moisture, soil water content, soil water potential and pH.

87. A probe for analysing a soil located in an underground area, the probe comprising:
a tubular body having a bottom portion and a top portion; and
at least one capacitive sensor, each capacitive sensor being mounted within the tubular body, in the bottom portion, and comprising:
a circuit board;
a signal generator operatively connected to the circuit board, the signal generator being configured to produce a plurality of driving signals, each driving signal having a central frequency comprised in a range extending from about 2 kHz to about 200 MHz;
an antenna wrapping an outer surface of the bottom portion of the tubular body and being operatively connected to the circuit board and to the signal generator, the antenna being electromagnetically coupled with the soil when the probe is inserted in the underground area and being configured to produce an electric field upon reception of one of said plurality of driving signals, the antenna comprising a ground coil and a signal coil adapted to provide a differential measurement; and
a measuring unit operatively connected to the antenna and being configured to determine a capacitance of the soil, based on a collection of differential measurements obtained at different frequencies, the capacitance of the soil being representative of at least one characteristic of the soil.

88. The probe of claim 87, wherein said at least one capacitive sensor is a stack of capacitive sensors.

89. The probe of claim 88, wherein two subsequent capacitive sensors of the stack of capacitive sensors are separated by a distance of about 6 inches.

90. The probe of claim 87, further comprising a casing, wherein the antenna is entirely covered by the casing.

91. The probe of claim 87, wherein the ground coil surrounds at least one of the circuit board and the signal generator.
